(19) United States
(12) Patent Application Publication
(10) Pub. No.: US 2012/0116275 A1
(43) Pub. Date: May 10, 2012

(54) Convertible Walker Boot and Post-op Shoe

(76) Inventor: David Pochatko, Williamsville, NY (US)

(21) Appl. No.: 12/943,232

(22) Filed: Nov. 10, 2010

(51) Int. Cl. A61F 5/00

(52) U.S. Cl. 602/13; 602/27

(57) ABSTRACT

A convertible walker boot, comprising a base with at least one fastener for securing a post-op shoe, the post-op shoe having an upper and a flexible sole having an opening for removably attaching at least one rigid member for adjusting rigidity, the post-op shoe having at least one fastener for securing the foot of a user, at least one brace having an upper member and a lower member, wherein the upper member is detachable from the lower member, wherein the base has a raised lip having at least one mounting slot for removably mounting the at least one brace to the base, wherein the at least one brace has at least one fastener for securing the leg of a user, and a sole stiffener removably mounted on the flexible sole.

### FIELD OF THE INVENTION

The present disclosure relates to orthopedic footwear devices, and more particularly to a convertible orthotic support which can be converted from a walking boot to a post-op shoe for assisting in the stabilization and proper healing of the foot.

### BACKGROUND OF THE INVENTION

Often times, when a person sustains an injury to or has surgery on their foot or ankle, the recovery process involves multiple stages requiring multiple different footwear devices. In the initial stages, the patient may need a high ankle walker (boot) which is very rigid, in order to eliminate any movement of the bones and joints in the foot. As the healing process progresses, the patient may then require a low ankle walker (semi-boot) with moderate flexibility, before finally stepping down to a flexible post-op shoe. Depending on the patient's injury or type of surgery, the patient may require other combinations of boots and shoes having dimensions and leg support with various degrees of flexibility.

Many people who have an injury, condition, or surgery to or on their feet or ankles will need a shoe that can be rigid and/or flexible during their recovery period. For example, those recovering from surgery to the foot often need a rigid full-length boot to eliminate movement of the bones and joints of the foot either in the immediate post-operative period or immediately subsequent to removal of a cast. Following that first period, it is frequently necessary and/or desirable to provide a shoe which will allow some movement of the bones and joints of the foot to encourage recovery. However, as the foot is often still swollen and sore during this second, later period, it is undesirable to allow the patient to wear his or her own shoes. Therefore, it is common that patients will cut the toe out of their own shoe, or worse, the patient will jam a swollen foot into their own shoe. In many instances the patient will purchase a single footwear device for use throughout all stages of recovery, thereby hindering the recovery process. This problem is exacerbated by common medical insurance coverage, which usually covers the cost of only one footwear device, as opposed to the multitude of devices a patient needs to promote proper healing following injury.

Several devices have been invented which have attempted to solve the problem of providing an orthosis that provides support and aid throughout the recovery process, but none have solved the problem. For example U.S. Pat. No. 6,648,843 (Marciano, et al.) discloses a combination night splint and walking boot. The night splint portion contains a thin sole that when separated from the boot is not suitable for walking. Although this patent teaches the desirability of a lightweight night splint which is modified to permit the patient to become ambulatory, it fails to provide convertibility to a low ankle supporting shoe from a full leg cast.

U.S. Pat. No. 7,418,755 (Bledsoe et al.) discloses a walking boot that promotes the healing of diabetic foot ulcerations. The boot has a modular design comprising a hard shell, a sole and protective bootie. The boot is not convertible to a shoe, nor is the sole of a rigid or flexible nature. U.S. Pat. No. 5,913,841 (William D. Lamont) discloses a medical boot which includes a lightweight night splint device and which includes a durable fabric material which is temporarily attached to the bottom of the boot to permit some ambulation on the part of the patient. Similarly, U.S. Pat. No. 5,609,570 issued to the same inventor, discloses a boot, which is wrapped around the night splint and patient's ankle to permit the user to ambulate while wearing a night splint. It should be apparent that while providing a solution to the problem of ambulation while wearing a lightweight night splint, neither of these designs provides anywhere near the weight-bearing durability of typical walking boot splints and consequently, it would be desirable to provide a method or system whereby a full leg walking boot is efficiently converted to a shoe without undue complexity.

Other braces provide varying ranges of support for an injured leg. For instance, U.S. Pat. No. 7,618,388 (Chan) discloses a high-rigidity walker boot. The boot contains multiple supports that strengthen leg support and resist side force. This device is deficient from the point of view of a patient with a progressively healing leg because the walker boot is not convertible to a shoe, nor does it have a rigid or flexible nature.

Another type of brace is shown in U.S. Pat. No. 7,524,295 (Peters et al.) that discloses a convertible ankle brace, able to function as a splint or as a non-restrictive brace, but is not designed to be worn as a post-op shoe.

Accordingly, it would be advantageous to provide a convertible walker/shoe that is used at various stages of treatment, from functioning as a high ankle walker (boot) when the leg is first injured, to functioning as a low ankle walker (semi-boot) as the leg begins to heal, to a relatively non-restrictive protective post-op shoe that provides different ranges of rigidity as the foot becomes completely healed. Thus it is readily apparent that there is a long felt need for a walker boot that is readily convertible to a post-op shoe to overcome these and other deficiencies in the art. The present disclosure satisfies the above-mentioned needs, as well as others, and overcomes the deficiencies in devices heretofore developed.

### SUMMARY OF THE INVENTION

It is a general object of the present disclosure to provide a convertible walker/shoe that is used at various stages of treatment, from functioning as a high ankle walker (boot) when the leg is first injured, to functioning as a low ankle walker (semi-boot) as the leg begins to heal, to providing a relatively non-restrictive protective post-op shoe that is easily convertible to different ranges of rigidity as the foot becomes completely healed.

A further object of the present disclosure is to provide an embodiment of the post-op shoe having a removable height adjusting brace.

It is another object of the present disclosure to provide a slidable or detachable, upright brace extension which attaches to a “static” (or functional) brace to provide an increased level of support and stability. With this upright extension, the brace is converted to a high ankle walker.

It is still another object of the present disclosure to provide a convertible walker boot/post-op shoe to allow the user to adjust the range of rigidity of the sole of the shoe to a multiplicity of settings in conjunction with the different height levels of the brace, to progressively treat an injury.

Another object of the present disclosure is to provide an embodiment with a slidable brace for slightly lowering the top portion of the brace to make it shorter and to provide for greater mobility once an injury begins to heal.

A further object of the present disclosure is to provide an embodiment with a detachable brace for removing the top portion of the brace to make it shorter and to provide for greater mobility once an injury begins to heal.

Still another object of the present disclosure is to provide an embodiment with a brace that is hinged at the ankle to provide for greater mobility once an injury begins to heal.

Another object of the present disclosure is to provide an embodiment with a pneumatic air bladder system which can inflate around the foot and/or ankle, providing for compression that is customized to the user.

The device of the present invention and its use is readily understood with reference to its use as a convertible walker boot, comprising a shoe support base for supporting a post-op shoe, at least one brace removably mounted to the shoe support base and a means for removably attaching the post-op shoe to the shoe support base. The brace can be adjusted to different heights as suited thereto or removed from the shoe and the shoe can be worn without the brace and provide a modicum of support. Medical walker boots are commonly made to immobilize a person's leg without much concern for adjustability as the leg heals. In a preferred embodiment, for use with legs in different stages of healing, the removable brace of the present device is a brace with a slidable extension. The brace is preferably positioned proximate the ankle portion of the post-op shoe. The shoe is preferably configured to cooperate with the brace to sufficiently stabilize the leg. When attached to the post-op shoe, the brace should extend a suitable distance beyond the sole to support the entire leg and the brace extension is adjustable to decrease the support as the leg heals.

These and other objects, features, and advantages of the present invention will become apparent upon a reading of the detailed description and claims in view of the several drawing figures.

### BRIEF DESCRIPTION OF THE DRAWINGS

The disclosure is explained below by means of schematic drawings (see attached) and with additional details. The drawings include:

FIG. 1 is a perspective view of the walker boot connected to the post-op shoe according to the disclosure.

FIG. 2 is an exploded view of the convertible walker boot according to the disclosure.

FIG. 3 is a broken-away view of the walker boot brace, set at high-ankle level, of the walker boot according to the disclosure.

FIG. 4 is a broken-away view of the walker boot brace, set at low-ankle level, of the walker boot according to the disclosure.

FIG. 5 is a broken-away view of the walker boot brace showing the detachable nature of the brace assembly according to the disclosure.

FIG. 6 is a perspective view of the post-op shoe secured to the support base of the walker boot according to the disclosure.

FIG. 7 is a perspective view of the rigid member of the post-op shoe according to the disclosure.

FIG. 8 is an exploded view of the rigid member assembly of the post-op shoe according to the disclosure.

FIG. 9 is a perspective view of the sole stiffener of the post-op shoe according to the disclosure.

FIG. 10 is a perspective view of the post-op shoe portion of the convertible walker boot according to the disclosure.

FIG. 11 is a broken-away view of the post-op shoe portion, without any rigid member, of the convertible walker boot according to the disclosure.

FIG. 12 is a broken-away view of the post-op shoe portion, showing flexibility of the sole without any rigid member, of the convertible walker according to the disclosure.

FIG. 13 is a perspective view of the post-op shoe portion and the rigid member assembly of the convertible walker boot according to the disclosure.

FIG. 14 is a perspective view of post-op shoe portion, with the rigid member assembly secured, of the convertible walker boot according to the disclosure.

FIG. 15 is a broken-away view of the post-op shoe portion, with the rigid member assembly secured, of the convertible walker boot according to the disclosure.

FIG. 16 is a broken-away view of the post-op shoe portion of the convertible walker boot, showing limited flexibility of the sole with the rigid member assembly secured according to the disclosure.

FIG. 17 is a perspective view of the post-op shoe portion of the convertible walker boot showing the removability of the sole stiffener according to the disclosure.

FIG. 18A is a broken-away view of the post-op shoe portion, with the sole stiffener and rigid member assembly secured, of the convertible walker boot according to the disclosure.

FIG. 18B is a broken-away view of the post-op shoe portion, with the sole stiffener secured and without any rigid member, of the convertible walker boot according to the disclosure.

FIG. 19 is a perspective view of the post-op shoe portion of the convertible walker boot with unsecured support straps, according to the disclosure.

FIG. 20 is a perspective view of the post-op shoe portion of the convertible walker boot, with support straps secured according to the disclosure.

### DETAILED DESCRIPTION OF THE INVENTION

### Description of Walker Boot Convertible to Post-op Shoe Drawings

At the outset, it should be clearly understood that like reference numerals are intended to identify the same structural elements, portions, or surfaces consistently throughout the several drawing FIGS., as is further described or explained by the entire written specification of which this detailed description is an integral part. The drawings are intended to be read together with the specification and are to be construed as a portion of the entire “written description” of this invention as required by 35 U.S.C. §112.

Medical walker boots are commonly made to immobilize a person's injured leg without much concern for adjustability as the leg heals. The preferred embodiment of the present disclosure is a convertible walker boot configured for use with injured legs throughout different stages of healing. The convertible walker boot is configured with a brace assembly having a slidable and removable extension operatively arranged to provide different levels of support, and a post-op shoe configured to adjust to different levels of flexibility. The combination of the adjustable brace assembly and post-op shoe presents the user with a multiplicity of possible settings.

The device of the present invention and its use is readily understood with reference to its use as a convertible walker boot, comprising a shoe support base for supporting a removable post-op shoe, at least one brace removably mounted to the shoe support base and a means for removably attaching the post-op shoe to the shoe support base. The preferred embodiment of the instant disclosure includes a removable brace assembly having adjustable braces. Each brace has an extension that can be lowered, raised or removed to give the user a variety of levels of support. The braces provide different levels of support for an injured leg based upon the positioning of the height of the brace.

The post-op shoe is preferably configured to cooperate with the brace to provide for a multiplicity of different combinations of shoe flexibility and variable heights of the brace so as to sufficiently stabilize the leg at different stages of healing. When attached to the post-op shoe, the brace extends a suitable distance beyond the sole to support the entire leg and the brace extension is adjustable to decrease the support as the leg heals.

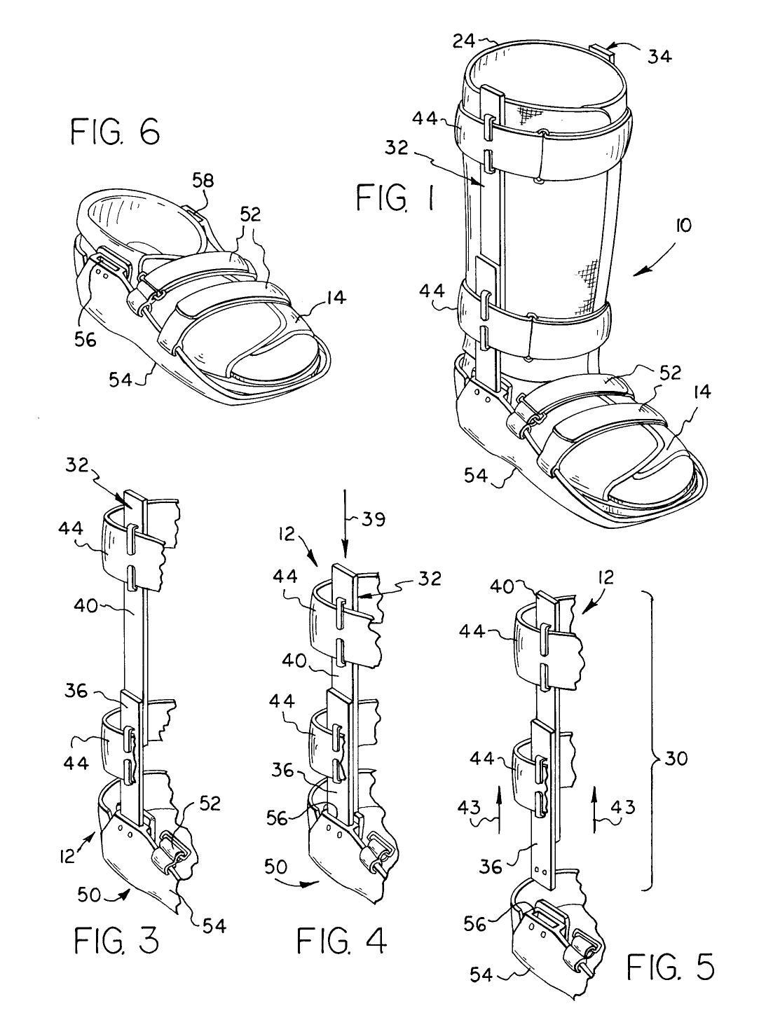

Averting now to the drawings, with reference to FIG. 1 is a perspective view of convertible walker boot 10 according to the disclosure comprising shoe support base 54 for supporting post-op shoe 14, and braces 32 and 34 removably mounted to the shoe support base. Brace assembly 30 (as shown in FIG. 5) includes both braces 32, 34 and leg brace straps 44. Post-op shoe 14 with liner 24 is removably inserted into the brace assembly 30, yielding a fully-functional convertible walker boot 10. The convertible walker boot is configured to secure a foot and leg into the device by tightening the shoe support straps 52 and leg brace straps 44.

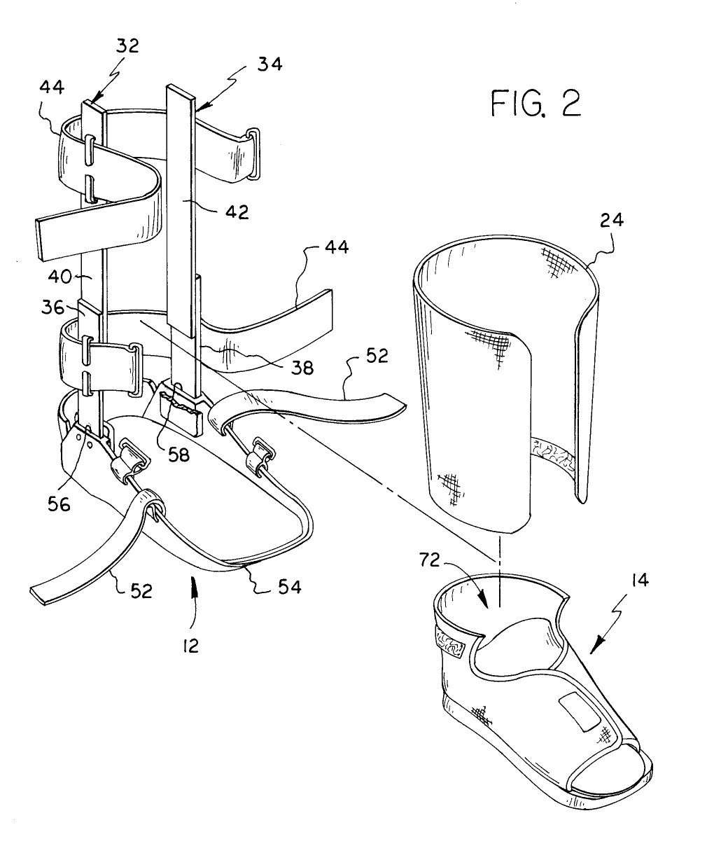

FIG. 2 shows an exploded view of convertible walker boot 10 according to the disclosure. In the preferred embodiment, liner 24 is removably attached to the rear of entry opening 72 of post-op shoe 14, as shown. Post-op shoe 14 is a shoe commonly used after an operation or shortly after an ankle or foot is injured. The shoe is configured with an open toe and adjustable flaps to comfortably secure the shoe to the foot and give support for walking yet not exacerbate an injury. Liner 24 attaches to either the inside or outside of post-op shoe 14, and is preferably made of any substantially rigid sheet foam or rubber like material. Liner 24 is preferably secured to post-op shoe 14 by hook-and-loop type fasteners, but is also secured by any number of fastening means, such as buckles, straps, laces, or clips. Liner 24 and post-op shoe 14 are removably connected to walker boot assembly 12 and brace assembly 30. Liner 24 provides additional support for a severely injured leg or ankle that needs a high level of support during the healing process. Depending on the injury of the user or stage of healing, liner 24 can be removed or added to the convertible walker boot.

In the preferred embodiment, walker boot assembly 12 is comprised of a walker boot brace assembly 30 (as shown in FIG. 5) and shoe support section 50 (as shown in FIGS. 3, 4). Support section 50 comprises support base 54 and a plurality of shoe support straps 52. Post-op shoe 14 mounts into shoe support section 50 as shown in FIG. 1, and is secured using a plurality of shoe support straps 52. In the preferred embodiment, the shoe support straps are comprised of nylon and are secured using hook and loop fasteners, but may be comprised of any type of strong, flexible material such as vinyl materials, plastics, cloths, or laces. It should be appreciated that other suitable materials, other than hook and loop fasteners can be used in the manufacture of the shoe support straps such as belts, buckles, laces, or clips. In a further embodiment, walker boot assembly 12 may contain a hinged ankle, allowing for additional movement. Another preferred embodiment of the disclosure is a plastic shell in place of the walker boot brace assembly 30, while still another embodiment includes a brace assembly 30 with an air bladder system that allows the user to customize the compression levels around the foot or ankle.

FIGS. 3, 4 and 5, are broken-away views of the boot assembly 12 (walker boot brace assembly 30 and shoe support section 50) of convertible walker 10 according to the disclosure. FIG. 3 depicts the walker boot brace assembly 30 set to a high-ankle position, while FIG. 4 depicts the walker boot brace assembly 30 set to a low-ankle level. The walker boot brace assembly 30 is comprised of an adjustable leg brace 32 and an identical second adjustable leg brace 34 and leg brace straps 44. FIG. 2 shows where each adjustable leg brace 32, 34 detachably engages a respective shoe support mounting slot 56, 58 in a position vertical to shoe support base 54. Each adjustable leg brace 32, 34 is comprised of a static member 36, 38 and a slidable member 40, 42, respectively.

FIG. 4 depicts how slidable member 40 of adjustable leg brace 32 is set to a low-ankle level by sliding it down toward shoe support section 50 in the general direction of arrow 39. Static members 36, 38 make up the lower portion of adjustable leg braces 36, 38 and include a means for detachably engaging shoe support mounting slots 56, 58. In the preferred embodiment, the means for detachably engaging static members with shoe support mounting slots is a lockable clip which is operatively arranged to allow the sliding member to be positioned at different height levels; however the means for detachably engaging static members with shoe support mounting slots is any number of securable devices, including a latch, screw, knob, or buckle.

In the preferred embodiment, mounting slots 56, 58 are located on shoe support base 54, but in another embodiment of the disclosure mounting slots are located on post-op shoe 14. Slidable members 40, 42 comprise the upper portion of adjustable leg braces 36, 38 and allow the user to adjust the height of brace assembly 30 from a high-ankle position to a low ankle position and vice-versa. In another preferred embodiment of the disclosure, slidable members 40, 42 are detachable from static members 36, 38, while still other embodiments have different methods of engagement between static members 36, 38 and slidable members 40, 42, such as through telescoping. Slidable members 40, 42 (whether detachable or not) allow the user a variety of different levels of support during the healing process.

Convertible walker boot 10 satisfies an individual's healing needs by combining specific levels of support (by adjusting the height level of the braces) with the different levels of post-op shoe flexibility (by changing the rigid member or rigid sole in the post-op shoe). By adjusting the height of the braces and the shoe rigidity there are many different combinations available to the user. For example, the braces provide varying degrees of support by raising or lowering the braces while the post-op shoe can be adjusted to have varying degrees of flexibility by removing the rigid member from the sole of the shoe.

FIG. 5 depicts boot assembly 12 according to the disclosure, with walker boot brace assembly 30 detached from mounting slot 56. Brace assembly 30 is removed from the shoe support section by pulling the braces away from the shoe support section generally in the direction of arrows 43. FIG. 6 illustrates a perspective view of the post-op shoe 14 secured to the shoe support base 54 of convertible walker 10 according to the disclosure. Post-op shoe 14 is secured to shoe support base 54 by means of the shoe support straps 52. Using a post-op shoe in conjunction with the shoe support base without any braces is beneficial if the user requires a maximum amount of rigidity in the foot, but does not require brace assembly 30 to restrict movement of an injured ankle or support an injured leg. In a further embodiment, shoe support straps 52 attach directly to post-op shoe 14 for securing a foot to the shoe, and the shoe attaches to support base 54 through a latch, clip, or other attachment means standard in the art.

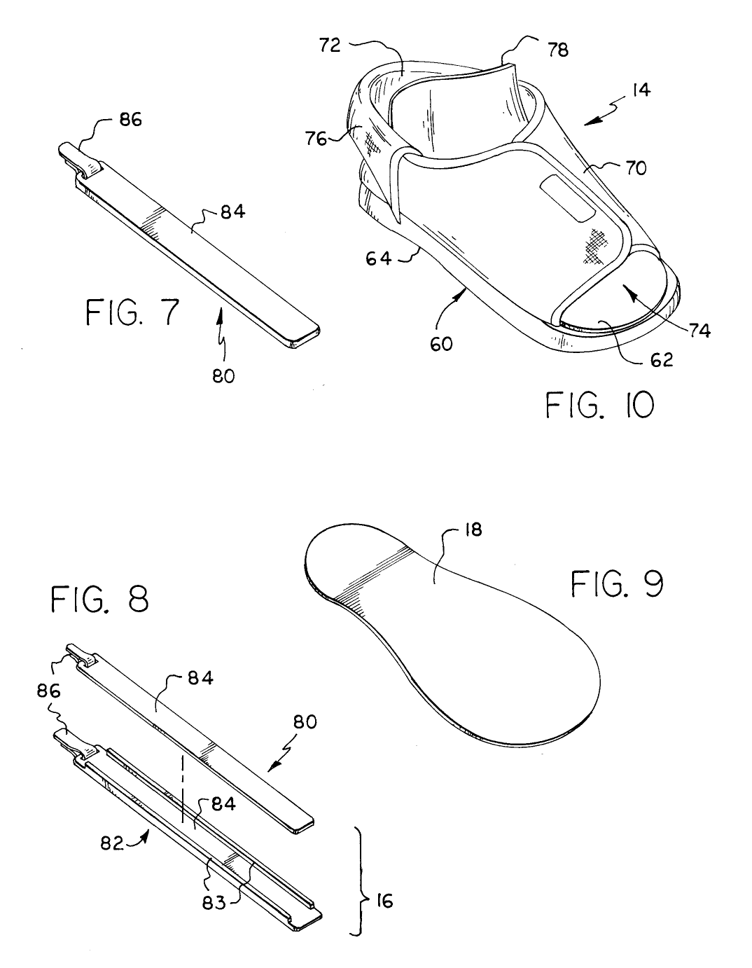

FIG. 7 shows a perspective view of rigid member 80 of convertible walker 10 according to the disclosure. Rigid member 80 is made of any suitable rigid material, including, but not limited to, carbon fiber or carbon fiber-type materials, steel, fiberglass, plastics, and any combinations thereof. Rigid member 80 includes shank 84 and attachment means 86. Shank 84 is a removable insert to adjust the rigidity and flexibility of the post-op shoe. Shank 84 is inserted into opening 66 within flexible sole 60 (as shown in FIG. 13), causing flexible sole 60 to become rigid. Rigid member 80 is secured within the opening 66 by attachment means 86, which is comprised of hook and loop type fasteners configured to secure rigid member 60 within the cavity, and act as a grip to allow the user to secure and remove the rigid member from the sole of the shoe. FIG. 8 is an exploded view of rigid member assembly 16. In this embodiment, rigid member assembly 16 is comprised of a rigid member 80 and a second rigid member 82. Second rigid member 82 has retaining lips 83 for slidably engaging first rigid member 80. The first and second rigid members each have different levels of rigidity and can be used alone or together in conjunction with the sole of the post-op shoe. The user has the ability to decrease the level of rigidity of the sole of the post-op shoe by removing all or part of the rigid member assembly 16. Rigid member assembly 16 is used to achieve a high level of rigidity in the sole 60 of the post-op shoe 14. Each rigid member has a different level of rigidity so that each member used separately or together to allow for still another level of rigidity or flexibility. This allows the user to utilize multiple degrees of rigidity, and to increase or decrease the rigidity levels as needed. Another preferred embodiment of the disclosure is a plate to be used in place of a shank.

FIG. 9 is a perspective view of the sole stiffener 18 of convertible walker 10 according to the disclosure. The sole stiffener 18 is shaped to fit within the upper 70 and on top of the insole 62 (shown in FIGS. 18A and 18B). As insole 62 is generally comprised of a relatively soft and flexible material, the sole stiffener 18 is added in the event the user requires additional support, and is used to adjust the flexibility of post-op shoe 14 from semi-rigid if no rigid members 80, 82 are used, to extra-rigid when used in conjunction with rigid members 80, 82. The post-op shoe is thus configured to provide multiple levels of rigidity that complement the multiplicity of support options of the brace assembly. Used in combination the instant invention provides for almost any setting of rigidity and support to aid in the healing process of an injured leg, foot, or ankle.

FIG. 10 depicts a perspective view of post-op shoe portion 14 of convertible walker 10 according to the disclosure. Post-op shoe 14 includes upper section 70 and flexible sole 60. In the preferred embodiment, the upper encapsulates the foot of the user by overlapping on the top portion of the foot. This overlapping allows the user to adjust the fit of post-op shoe 14 throughout the healing process as swelling increases or decreases. The overlapping part of upper 70 is secured by hook and loop type fasteners (or by another fastening means). In other preferred embodiments of the disclosure, upper 70 overlaps at a different portion of the foot. In another embodiment, upper 70 is comprised of a continuous sheet of material and therefore there is no overlap. It should be understood by one skilled in the art that upper 70 is preferably made of a soft, flexible material such as leather, synthetic, vinyl, cloth, plastic, or a combination thereof.

Upper section 70 includes an entry opening 72 for the foot and open toe 74. Open toe 74 allows for the shoe to be more easily placed on the user by providing the foot additional room, accounting for swelling during the healing period, and alleviating pressure on the foot of the user. Tongue 78 is removably attached to upper 70 and is located adjacent to the entry opening 72. Tongue 78 is adjusted along upper 70 to increase or decrease the size of the entry opening, thereby helping to encapsulate the foot completely, as well as adding to the comfort of the user. In the preferred embodiment, tongue 78 is removably attached to upper 70 by hook and loop type fasteners. In another embodiment, the tongue 78 is permanently attached to the upper 70. When liner 24 is not connected to post-op shoe 14, collar 76 is formed adjacent to entry opening 72 by folding down the portion of upper 70 that the liner removably attaches to. The additional layer of material formed by collar 76 increases support to the foot of the user.

Flexible sole 60 of the post-op shoe is comprised of insole 62, outsole 64, and an opening 66. In the preferred embodiment, flexible sole 60 is substantially planar, and is generally shaped to accommodate either the left foot or the right foot comfortably. In other embodiments, a non-planar sole is used (e.g. with arch support). In still further embodiments, the flexible sole section is shaped specifically to accommodate either the left foot or the right foot. The flexible sole section is attached to the upper section using suitable techniques known in the art, such as through adhesives or stitching.

Outsole 64 of flexible sole 60 is made of a durable and flexible material, allowing post-op shoe 14 to partially bend yet not collapse. The bottom of outsole 64 is suitable for making contact with the ground and includes a tread surface. It should be appreciated that suitable materials for outsole 64 include rubber or rubber-like materials, leather, vinyl materials, injection-molded materials, wood or wood-like materials, ethyl vinyl acetate (EVA), polyurethanes and the like.

Insole 62 of flexible sole 60 is located on top of outsole 64 and within upper 70. In the preferred embodiment, the purpose of insole 62 is to provide for the comfort of the user, and insole 62 does not add to the supportive structure of the shoe. However, other embodiments allow for insole 62 to add to the support structure of the shoe. The insole 62 is generally comprised of a relatively soft and flexible pad which will conform to the foot of the user.

Referring now to FIG. 11, there is depicted a broken-away view of the post-op shoe 14, without any rigid member 80, of convertible walker 10 according to the disclosure. Opening 66, located within the flexible sole 60 between insole 62 and outsole 64, extends longitudinally from heel 68 of post-op shoe 14 to toe 69 of the post-op shoe 14. In the preferred embodiment, opening 66 is of suitable shape and size to receive either a single rigid member 80, 82, or rigid member assembly 16.

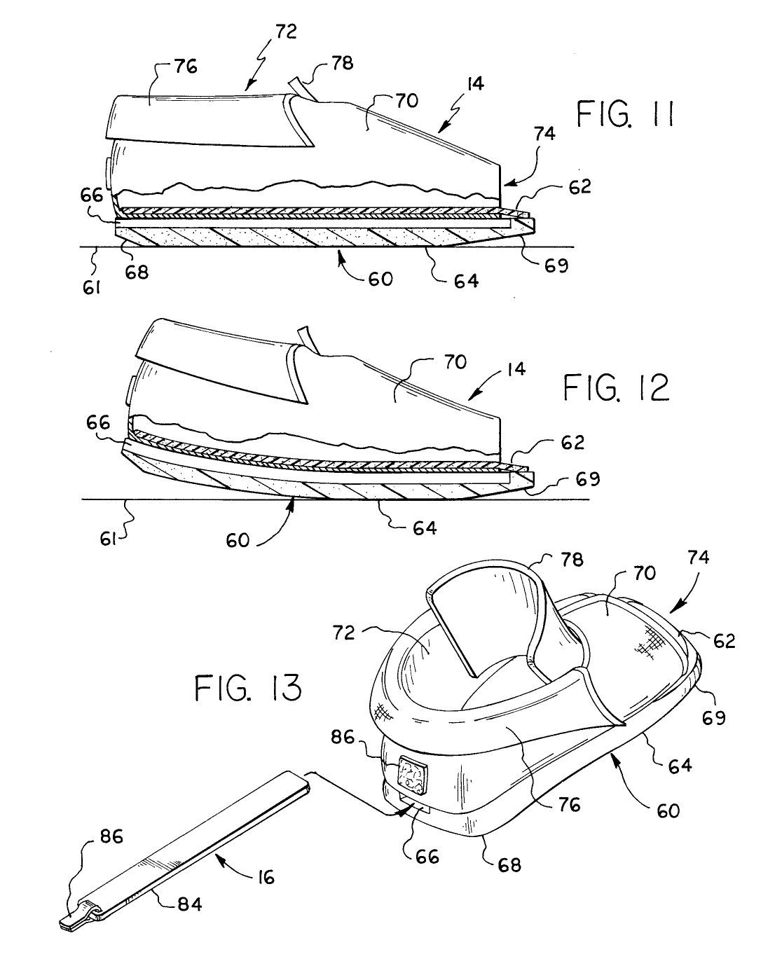

With reference now to FIG. 12, there is depicted a broken-away view of the post-op shoe 14, showing flexibility of the sole 60 without any rigid member 80, of convertible walker 10 according to the disclosure. FIG. 12 depicts the flexible nature of the post-op shoe 14 when there is no rigid member in opening 66 of the flexible sole 60. As shown in FIG. 12, when the shoe is used in walking the sole flexes with the foot. The sole is shown in FIG. 12 flexed away from ground 61.

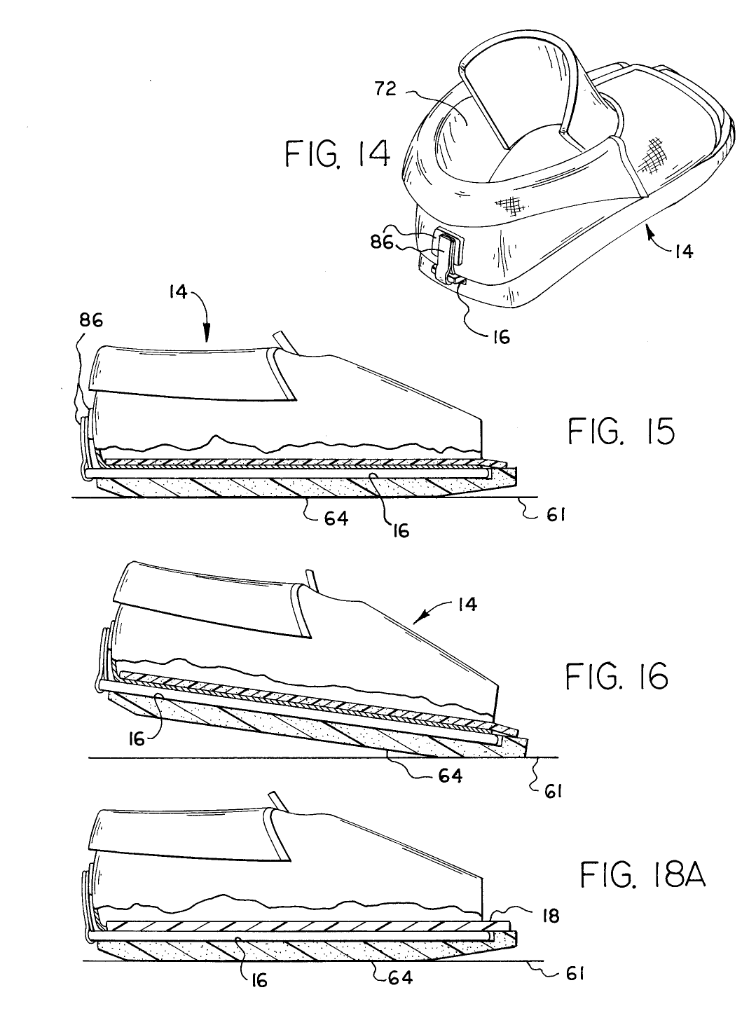

Referring now to FIGS. 13 through 15, there is depicted the post-op shoe 14, with rigid member assembly 16 inserted and secured in opening 66, of convertible walker 10 according to the disclosure. FIG. 13 shows rigid member assembly 16 lined up with opening 66 of the flexible sole 60, ready to be inserted by the user. In the preferred embodiment, the rigid member assembly 16 is inserted into the opening 66 and is secured to the upper 70 through attachment means 86 which are hook and loop type fasteners, as shown in FIG. 14. In additional embodiments rigid member assembly 16 is secured through any number of fastening means, as described above.

FIG. 15 depicts a broken-away view of post-op shoe 14, with rigid member assembly 16 secured. Rigid member assembly 16 extends from opening 66 at heel 68 to toe 69 of post-op shoe 14.

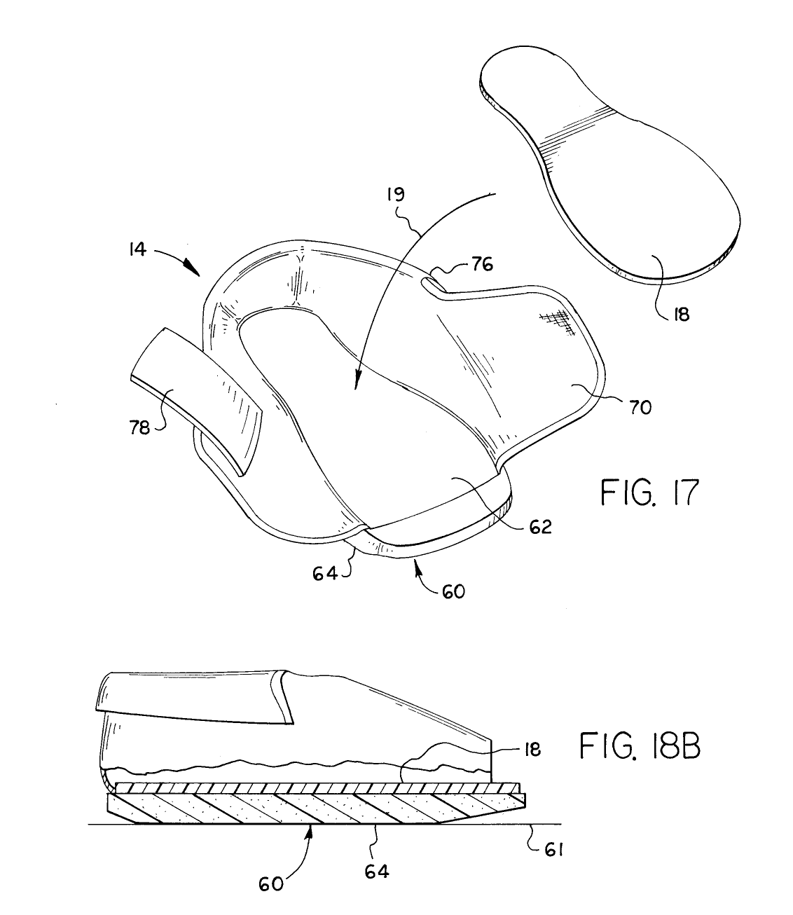

FIG. 16 depicts a broken-away view of post-op shoe 14, showing limited-to-no flexibility of sole 60 with the rigid member assembly 16 secured. Flexible sole 60 has become rigid by means of the insertion of the rigid member assembly 16. When the shoe is worn and lifted away from ground 61 the sole remains static and inflexible.

Referring now to FIG. 17, there is depicted a perspective view of the sole stiffener 18 being placed generally along arrow 19 into post-op shoe 14 of convertible walker 10 according to the disclosure. In the preferred embodiment of the disclosure, upper 70 of post-op shoe 14 is opened allowing for sole stiffener 18 to be easily placed directly onto insole 62. In another preferred embodiment of the disclosure, upper 70 of post-op shoe 14 does not open and sole stiffener 18 is inserted into upper 70 through the entry opening 72 (as shown in FIG. 14). Still other preferred embodiments allow for sole stiffener 18 to be placed in between insole 62 and outsole 64.

FIGS. 18A and 18B depict broken-away views of post-op shoe 14 of convertible walker 10 according to the disclosure. FIG. 18A depicts post-op shoe 14 with both rigid member assembly 16 and sole stiffener 18 secured. FIG. 18B depicts the post-op shoe 14 with the sole stiffener 18 secured but without the rigid member assembly 16.

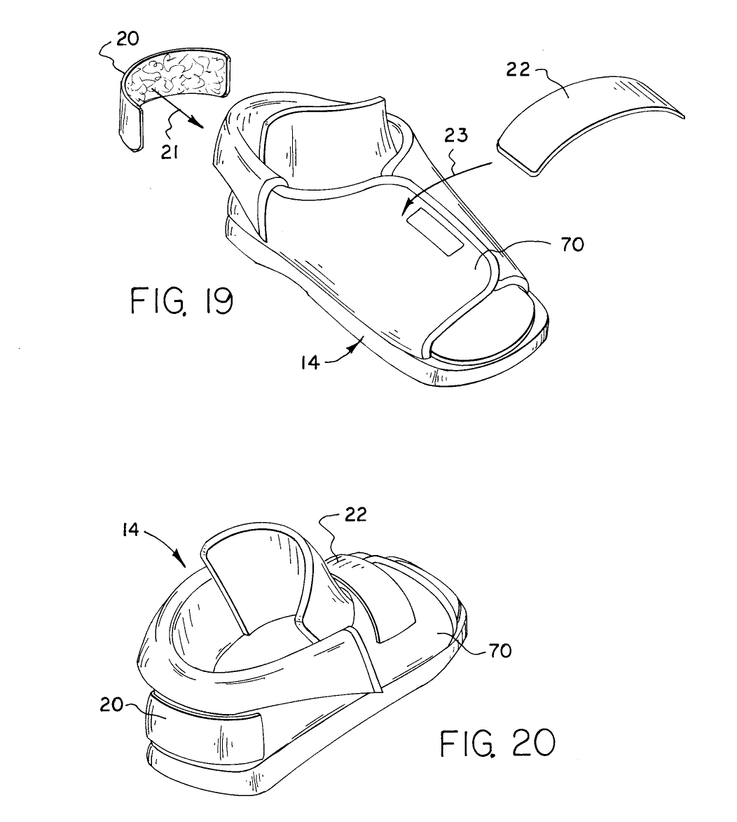

With reference to FIGS. 19 and 20, there is depicted a perspective view of the post-op shoe 14 of convertible walker 10 with heel support 20 and foot support 22 attached according to the disclosure. Depending on the nature of the recovery, a user of convertible walker 10 may wish for post-op shoe 14 to have additional heel support. FIG. 19 depicts heel support 20 being placed along the back of upper 70 of the post-op shoe 14 generally in the direction indicated by arrow 21, as well as foot support 22 placed along the top of upper 70 of post-op shoe 14 generally in the direction indicated by arrow 23. FIG. 20 depicts heel support 20 and foot support 22 secured to post-op shoe 14. The addition of one or both supports serves to restrict the movement of the foot when walking. It should be understood that heel support 20 and foot support 22 are made of any substantially rigid material, and are secured to the post-op shoe 14 through a number of different fastening means, as described above.

While a preferred form of this disclosure has been described above and shown in the accompanying drawings, it should be understood that applicant does not intend to be limited to the particular details described above and illustrated in the accompanying drawings, but intends to be limited only to the scope of the invention as defined by the following claims. In this regard, the term “means for” as used in the claims is intended to include not only the designs illustrated in the drawings of this application and the equivalent designs discussed in the text, but it is also intended to cover other equivalents now known to those skilled in the art, or those equivalents which may become known to those skilled in the art in the future.

What is claimed is:

1. A convertible walker boot, comprising:
a base for holding a post-op shoe,
at least one brace removably mounted to said base for providing support;
a means for adjusting said support of said convertible walker boot;
a means for adjusting the rigidity of said post-op shoe; and
a means for removably attaching said post-op shoe to said base.

2. The convertible walker boot of claim 1, wherein said post-op shoe comprises:
an upper and a flexible sole with an opening for removably attaching at least one rigid member.

3. The convertible walker boot of claim 2, wherein said at least one rigid member is a rigid member assembly that can be separated for achieving multiple levels of rigidity.

4. The convertible walker boot of claim 1, wherein said at least one brace comprises an upper member and a lower member.

5. The convertible walker boot of claim 4, wherein said upper member slides adjacent to said lower member for adjusting the height of said at least one brace.

6. The convertible walker boot of claim 4, wherein said upper member is detachable from said lower member.

7. The convertible walker boot of claim 1, wherein said base has a raised lip having at least one mounting slot for removably mounting said at least one brace to said base.

8. The convertible walker boot of claim 1, wherein said at least one brace is removably attached to said base by a hinge.

9. The convertible walker boot of claim 1, further comprising a pneumatic air bladder for customizing the pressure around the foot or leg of a user.

10. The convertible walker boot of claim 1, wherein said at least one brace includes at least one fastener for securing the leg of a user.

11. The convertible walker boot of claim 1, wherein said base includes at least one fastener for securing said post-op shoe.

12. The convertible walker boot of claim 2, further comprising a sole stiffener removably mounted on said flexible sole.

13. The convertible walker boot of claim 2, wherein said at least one rigid member is made of a material selected from the group consisting of carbon fiber, steel, fiberglass, and plastics.

14. The convertible walker boot of claim 2, wherein said upper is made of a material selected from the group consisting of leather, vinyls, plastics, and cloths.

15. The convertible walker boot of claim 2, wherein said flexible sole is made of a material selected from the group consisting of rubbers, vinyls, leather, injection-molded materials, wood, ethyl vinyl acetate, and polyurethanes.

16. A convertible walker boot, comprising:
a base with at least one fastener for securing a post-op shoe having an upper and a flexible sole having an opening for removably attaching at least one rigid member for adjusting rigidity, said post-op shoe having at least one fastener for securing the foot of a user;
at least one brace having an upper member and a lower member, wherein said upper member is detachable from said lower member, wherein said base has a raised lip having at least one mounting slot for removably mounting said at least one brace to said base, and wherein said at least one brace has at least one fastener for securing the leg of a user; and
a sole stiffener removably mounted on said flexible sole.

17. A convertible walker boot, comprising:
a boot having a base, at least one brace removably connected to said base, and a post-op shoe removably connected to said base; and
a means for adjusting said boot between a high-ankle walker and a low-ankle walker.

18. The convertible walker boot of claim 17, wherein said at least one brace comprises an upper member and a lower member, and wherein said means for adjusting said boot between a high-ankle walker and a low-ankle walker is said upper member sliding up and down adjacent to said lower member.

19. The convertible walker boot of claim 17, wherein said at least one brace comprises an upper member and a lower member, and wherein said means for adjusting said boot between a high-ankle walker and a low-ankle walker is said upper member is removable from said lower member.
